(19) United States
(12) Patent Application Publication
(10) Pub. No.: US 2015/0183696 A1
(43) Pub. Date: Jul. 2, 2015

(54) Naphthalene-Depleted Fluids

(71) Applicant: ExxonMobil Chemical Patents Inc., Baytown, TX (US)

(72) Inventors: Thomas A. Gribschaw, Friendswood, TX (US); Brian W. Roberts, Prairieville, LA (US); Andrew R. Witt, Huffman, TX (US)

(73) Assignee: ExxonMobil Chemical Patents Inc., Baytown, TX (US)

(21) Appl. No.: 14/405,710

(22) PCT Filed: Sep. 16, 2013
(86) PCT No.: PCT/US2013/059916

§ 371 Date: Dec. 4, 2014

Related U.S. Application Data
(60) Provisional application No. 61/703,300, filed on Sep. 20, 2012.

(30) Foreign Application Priority Data
Nov. 28, 2012 (EP) 12194520.8

(51) Int. Cl. C07C 7/04; B01D 3/32; B01D 3/14; C07C 7/00; C07C 15/24

(52) U.S. Cl. CPC C07C 7/04; C07C 7/005; C07C 15/24; B01D 3/143; B01D 3/32

(57) ABSTRACT

The invention relates to the production of naphthalene-depleted heavy aromatics, the naphthalene-depleted heavy aromatic product made thereby, and to an apparatus adapted for said production.

### PRIORITY CLAIM

This application claims priority to and the benefit of U.S. Provisional Application No. 61/703,300, filed Sep. 20, 2012, the disclosure of which is incorporated herein by reference in its entirety.

### FIELD OF THE INVENTION

The invention relates to the production of naphthalene-depleted heavy aromatics, the naphthalene-depleted heavy aromatic product made thereby, and to an apparatus adapted for said production.

### BACKGROUND OF THE INVENTION

Naphthalene-depleted aromatic fluids are gaining in importance and increased supply of such fluids is needed. Traditional fractionation schemes to produce such fluids involve sequencing fractionation towers to remove light cuts, as shown in FIG. 1. FIG. 1 is a schematic illustrating conventional production of useful fluids, including naphthalene-depleted fluids, from reformate. By way of example, topped reformate 1 is introduce into the first tower 2, and by progression through a second tower 5, third tower 8, and fourth tower 11, fluidly connected through conduits 4, 7, and 10, respectively, leading to recovering naphthalene as an overhead stream 9 and useful products such as 3 Aromatic 100 Fluid (CAS Registry No. 64742-95-6; distillation range of 161 to 171° C.; Flash Point 46° C.; 99.5 wt % aromatics), 6 Aromatic 150 ND Fluid (CAS Registry No. 70693-06-0; distillation range of 184-195° C.; Flash Point 65° C.; 99.7 wt % aromatics), naphthalene, 12 Aromatic 200 ND Fluid (CAS Registry No. 68477-31-6; distillation range of 235-278° C.; 106° C.; 99.5 wt % aromatics), and heavy byproduct 13. The distillation range is taken from the initial boiling point (IBP) to dry point (DP), terms defined further hereinbelow. Aromatic 100 Fluid, Aromatic 150 ND Fluid, Aromatic 200 ND Fluid are all well-known fluids available from ExxonMobil Chemical Company, Baytown, Tex., USA and/or Sarnia, Canada. The designation “ND” indicates naphthalene-depleted and, as used herein, the designation “ND” and the term “naphthalene-depleted” means a fluid having less than 1.0 wt % naphthalene content based on the total weight of the fluid. Topped reformate is reformate having BTX—benzene, toluene, and xylene—removed. The terms Initial Boiling Point (IBP) and Dry Point (DP) take their well-known meaning in the art and define the temperature range over which the fluid boils. For the purposes of the present invention, including claims, reference to IBP and DP are to measurements taken according to ASTM D86.

The production of the naphthalene-depleted products in the aforementioned manner is problematic since naphthalene's high freeze point results in additional steps to prevent plugging of tower overhead systems.

Solutions offered by the prior art include introduction of a diluent with the feed or in the tower overhead system resulting in energy inefficiencies and/or the warming cooling medium to assure temperatures are maintained above the freezing point. Specific examples of fractionation processes involving naphthalene include the following.

U.S. Pat. No. 3,193,952 teaches a method of making naphthalene wherein, in an embodiment, the product naphthalene is taken as a bottoms product but the overheads are recycled to extinction.

CN 1112539A discloses a three tower batch rectification for the production of isopropyl naphthalene. Tower I is used for dewatering and removal of naphthalene and monoisopropyl naphthalene. Tower II is for producing tri-isopropyl naphthalene and the tower III for producing fine di-isopropyl naphthalene.

JP 62167736A teaches naphthalene preparation from cracking gas, comprising (1) removing naphthalene-rich fraction as side-cut from gasoline fractionating tower, and (2) separating naphthalene from side-cut.

GB 2299342A teaches a process for efficiently controlling the bottom temperature of a primary fractionator column, particularly for the purpose of avoiding blocking by crystallization of diaromatics and more particularly naphthalene, comprising: (a) withdrawing at least one oil fraction stream from the primary fractionator column and cooling; (b) separating remaining liquid and precipitate; and (c) returning remaining liquid to fractionator.

JP 60013723A teaches that crude naphthalene oil is introduced in a column where it is subjected to atmospheric distillation; the bottom liquid is removed as a heavy oil, and vapour of the overhead fraction is fed via a partial condensation reboiler to a light oil separating column, where it is distilled under reduced pressure, and a light oil is recovered from the column top while naphthalene is recovered from the column bottom. The partial condensation reboiler condenses at least part of the overhead fraction from the heavy oil separating column so that the obtained heat may be used as a heat source for the light oil separating column. The residual constituent obtained when the vapour is partially condensed is fed to the light oil separating column.

The removal of naphthalene from a mixture comprising naphthalene and alkylbenzenes is disclosed in U.S. Pat No. 7,189,887. The process described therein comprises: feeding a stream containing naphthalene and alkylbenzenes to a distillation column reactor containing a bed of transalkylation catalyst and contacting the naphthalene with the alkylbenzenes in the presence of the alkylation catalyst and separating the unreacted alkylbenzenes from the unreacted naphthalene and alkyl naphthalene by fractional distillation. Naphthalene is said to accumulate in the reaction zone until it is converted to alkylnaphthalenes.

In U.S. Pat. No. 3,108,063, naphthalene is prepared from petroleum fractions by blending the reformate feedstock with the effluent from the hydrodealkylation reactor in the presence of a catalyst. The resulting mixture is separated into 4 fractions, the lightest being gasoline.

Naphthalene is separated from the second lightest cut by fractional crystallization and the other liquor recirculated to a hydrodealkylation zone. The third fraction is also recirculated to the hydrodealkylation zone. A fourth fraction is used for fuel oil.

JP 1198687A teaches recovering light oils and naphthalene, in a method comprising distilling an oil having absorbed the light oils and naphthalene from coke oven gas, in a tower containing a reboiler in the bottom.

JP 60067438A teaches a method involving: (a) directly introducing benzene-containing oil to a benzene removing tower without dehydration; (b) steam stripping with steam from the bottom while dehydrating; (c) refluxing the fraction from the middle stage of the benzene removing tower and recovering crude benzene from the top and crude naphthalene from the middle stage of the tower. The dehydrating tower used in known processes is not required.

JP 60013723 teaches separating crude naphthalene into a naphthalene product having a purity of at least 95 wt % and a light oil product.

An additional relevant reference is “Conserve heat consumption in naphthalene distillation”, Hydrocarbon Processing, April 2005 (pp. 55-60).

The present inventor has surprisingly discovered a simple but elegant method of avoiding prior art problems.

### SUMMARY OF THE INVENTION

The invention is directed to a method of providing naphthalene-depleted aromatic product, and to an apparatus adapted therefor. Fractionation towers and fluid connections are configured to provide a system that ultimately takes off naphthalene as a bottom stream, where it can be assured to be above its freezing point (80° C. for pure naphthalene), and at least one, and preferably plural naphthalene-depleted aromatic fluids are also taken off as products. Taking naphthalene off as a bottoms product also ensures a higher level of impurities, thereby depressing the freezing point and making it easier to avoid freezing of the bottoms product in the lines.

It is an object of the invention to provide a simple system involving fractionation towers to efficiently remove naphthalene from aromatic solvents, to provide naphthalene-depleted solvents, while avoiding prior art problems.

It is further an object of the invention to reconfigure existing fractionation towers to provide a more efficient system of providing naphthalene-depleted fluids.

It is yet another object of the invention to provide an apparatus that may be integrated by controllers for at least one of temperature, pressure, and heat integration so as to provide a range of naphthalene-depleted products.

These and other objects, features, and advantages will become apparent as reference is made to the following detailed description, preferred embodiments, examples, and appended claims.

### BRIEF DESCRIPTION OF THE DRAWINGS

In the accompanying drawings, like reference numerals are used to denote like parts throughout the several views.

FIG. 1 is a prior art configuration of plural towers to provide naphthalene-depleted solvents.

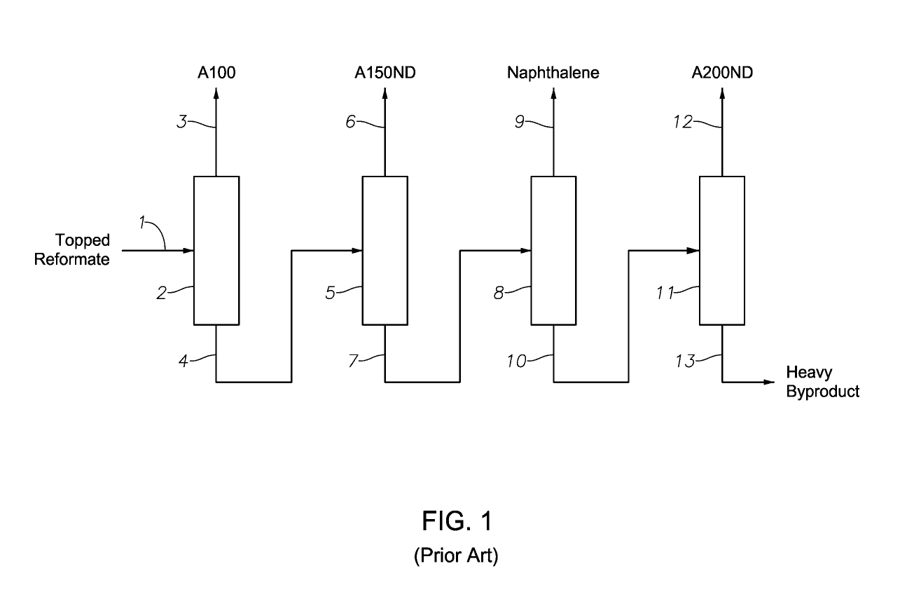

FIG. 2 is an embodiment of an apparatus according to the present invention, and adapted for the process of according to the present invention, to produce the product according to the present invention.

### DETAILED DESCRIPTION

According to the invention, towers and fluid connections associated therewith are arranged so that naphthalene is taken off as a bottoms product and at least one and preferably plural naphthalene-depleted aromatic fluids are also taken off By the expressions “taken off” and “taking off” it is meant that at least a portion of the identified product(s) is/are removed from the system.

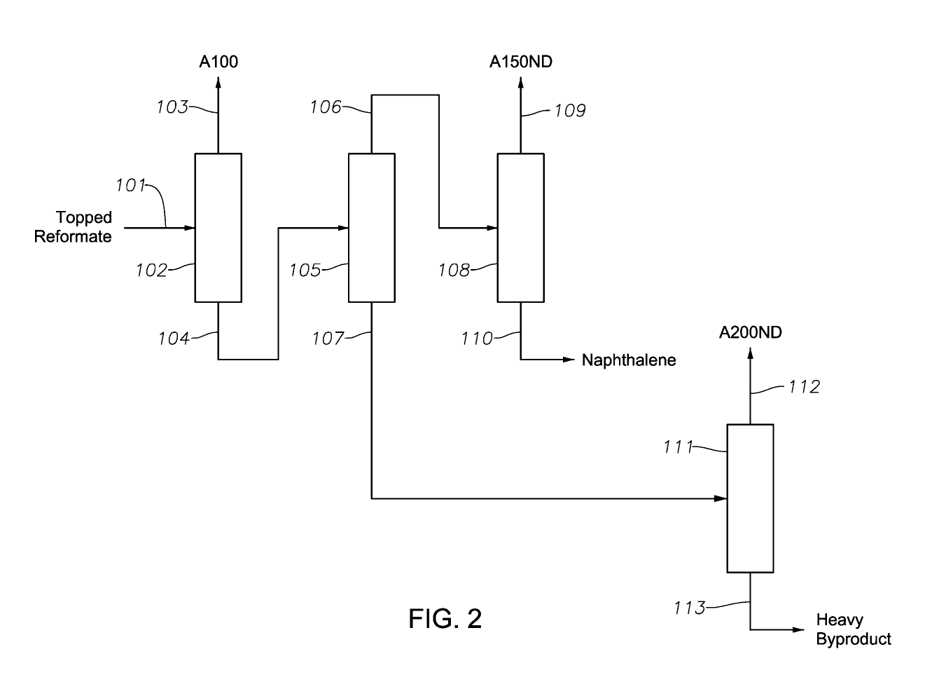

Typically four towers are arranged to process a reformate stream, the last two in parallel from a second tower, one tower to process the bottoms from said second tower and one tower to process the overheads. Naphthalene goes overhead from the second tower, and then is finally taken off as a bottoms product from the next tower in series (tower 3); a naphthalene-depleted heavy aromatics product (such as Aromatics 200 ND) is taken off as the overheads from tower 3. The naphthalene product taken off is typically less than 90 wt % napththalene, such as from 25-85 wt % or 30-70 wt % or 40-60 wt %, with ranges from any lower amount listed to any higher amount listed also being contemplated, e.g., 30-60 wt %. While higher purity naphthalene may be recovered by further processing out of the system shown in FIG. 2, below, conventionally the naphthalene recovered from this system is used as fuel.

The invention may be better understood by reference to FIG. 2, which is a schematic of a process according to the present invention, and an apparatus adapted therefor. It will be appreciated that the process and apparatus therefor may be practiced other than as specifically recited in the following specific example. Various valves, heat exchangers, and other details of such an apparatus are not shown for convenience of view, but would be apparent to one of ordinary skill in the art in possession of the present disclosure.

In FIG. 2, topped reformate 101 is provided to a fractionation tower 102 and a light aromatic hydrocarbon such as Aromatic 100 Fluid is taken off as overheads 103. The bottoms product 104 is passed to a second fractionation tower 105 and an overhead 106 is passed to a third fractionation tower 108, which in turns provides a naphthalene-depleted fluid taken off as overhead 109, such as Aromatic 150 ND Fluid, and naphthalene is taken off as a bottoms product 110; this is a highly advantageous aspect of the invention. The bottoms product 107 of fractionation tower 105 is passed to a fourth tower 111 to be separated into a heavy bottoms product 113 and an overhead 112, such as Aromatic 200 ND Fluid.

The system depicted in FIG. 2 nominally operates as a closed system. As such, tower 105 typically will be under a couple of pounds of pressure, such as from 1-6 psig (gauge pressure), or in embodiments, 3-6 psig (corresponding to about 0.0069-0.0414 Mpa-gauge, and 0.021 to 0.0414 Mpa-gauge, respectively). The temperature regime of tower 105, in advantageous embodiments, will range from 370 to 525° F. (188-274° C.) from the bottom of the tower to the top of the tower. In a preferred embodiment not shown, conduit 106 may pass through a cooler and the overhead split, with a portion continuing on to tower 108 and a portion being recycled to tower 105, and likewise conduit 107 may pass through a heater, with a portion of the bottoms product continuing on to tower 111 and a portion being recycled to tower 105.

One of skill in the art in possession of the present disclosure and the prior art cited in the Background section can modify the aforementioned, such as with controllers for at least one of temperature, pressure, and heat integration, and also by computer and/or operator assisted controls in order to maximize efficiency and operating characteristics of the system. All references recited herein are fully incorporated by reference to the extent such disclosure is not inconsistent with this invention and for all jurisdictions in which such incorporation is permitted.

We claim:

1. A process for producing naphthalene and at least one naphthalene-depleted fluid by use of an apparatus comprising plural fractionation towers, wherein naphthalene is taken off as a bottoms product from at least one of said plural fractionation towers and a naphthalene-depleted fluid having less than 1.0 wt % naphthalene is taken off as an overhead from at least one of said plural fractionation towers.

2. The process of claim 1, including the steps of passing a reformate stream to a first fractionation tower to separate said reformate stream into a first overhead product comprising an aromatic fluid and a first bottoms product comprising naphthalene, passing said first bottoms product to a second fractionation tower and taking a second overhead product comprising naphthalene, said process further characterized by passing said second overhead product to a third fractionation tower, and separating said second overhead product from said second fractionation tower into a third bottoms product comprising naphthalene is taken off and a third overhead product comprising a naphthalene-depleted aromatic fluid is taken off

3. The process of claim 2, wherein said reformate stream is topped reformate, characterized as depleted of benzene, toluene, and xylene by at least one step of fractionation downstream of a reforming unit.

4. The process of claim 1 any one of the preceding claims, including taking at least two overhead products from said apparatus, wherein one of said at least two overhead products has an initial boiling point (IBP) of about 188° C. and a dry point (DP) of about 200° C., and wherein the other of said at least two overhead products has an initial boiling point (IBP) of about 238° C. and a dry point (DP) of about 275° C., and wherein both of said at least two overhead products have a content of naphthalene of less than 1.0 wt %.

5. The process of claim 1 any one of the preceding claims, including taking an overhead from said apparatus having an initial boiling point (IBP) of about 161° C. and a dry point (DP) of about 171° C.

6. The process of claim 1 any one of the preceding claims, wherein said naphthalene taken off as a bottoms product has a purity of less than 90 wt % based on the weight of the product taken off as bottoms product.

7. The process of claim 6, wherein said naphthalene taken off as a bottoms product has a purity of from 30-60 wt %.

8. An apparatus comprising a source of reformate, a first fractionation tower comprising an overhead outlet and a bottoms outlet, said bottoms outlet of said first fractionation tower fluidly connected with a second fractionation tower, said second fractionation tower having an overhead outlet and a bottoms outlet, said overhead outlet of said second fractionation tower fluidly connected with a third fractionation tower and said bottoms outlet of said second fractionation tower fluidly connected with a fourth tower, and wherein each of said third fractionation tower and said fourth fractionation tower has an overhead outlet adapted for taking off fluids from said apparatus and a bottoms outlet, and wherein each of said first, second, third, and fourth fractionation towers have temperature controllers suitable for taking off overhead successively higher boiling aromatic fluids, respectively.

9. The process of claims 6, further characterized by controllers for at least one of temperature, pressure, and heat integration sufficient for taking naphthalene as a bottoms product from a fractionation tower in said apparatus and a naphthalene-depleted fluid from the same fractionation tower in said apparatus, wherein said naphthalene-depleted fluid is defined as comprising less than 1.0 wt % naphthalene.
